(19) United States
(12) Patent Application Publication
(10) Pub. No.: US 2010/0092564 A1
(43) Pub. Date: Apr. 15, 2010

(54) Composition of and Method for Preparing Orally Disintegrating Tablets

(76) Inventors: Jae Han Park, Olivette, MO (US); Stephen H. Wu, Chesterfield, MO (US); Kevin M. Holman, Lebanon, IL (US); Cliff J. Herman, St. Louis, MO (US)

Correspondence Address: Mallinckrodt Inc., 675 McDonnell Boulevard, HAZELWOOD, MO, 63042, US

(21) Appl. No.: 12/519,914

(22) PCT Filed: Dec. 20, 2007
(86) PCT No.: PCT/US07/26184

§ 371 Date: Jun. 18, 2009

Related U.S. Application Data
(60) Provisional application No. 60/876,383, filed on Dec. 21, 2006.
(60) Provisional application No. 60/948,208, filed on Jul. 6, 2007.

(51) Int. Cl. A61K 9/16; A61K 47/02

(52) U.S. Cl. 424/489; 514/769; 514/770

(57) ABSTRACT

An improved orally dissolving tablet (ODT) and method of manufacture is provided. The improved ODT disclosed herein are prepared by direct compression of a mixture of pharmaceutical excipients including at least one water-insoluble hydrophobic inorganic salt in combination with at least one water-insoluble inorganic salt with less hydrophobicity compared to the water-insoluble hydrophobic inorganic salt component. These components may be formed into granules, and may include other commonly used excipients. In an illustrative embodiment, the granules are formed into tablets by direct compression, optionally using a lubricant. The fast disintegrating tablets prepared using these components exhibit desirable performance properties such as sufficient hardness, low friability, quick disintegration time and good mouth-feel when compared to conventional ODT. A further advantage is that the improved ODT may be manufactured using commonly available manufacturing equipment for granulation, blending and tableting.

### BACKGROUND OF INVENTION

Conventional pharmaceutical oral dosage forms include tablets and capsules that are swallowed whole. Unfortunately, it has been estimated that 35% to 50% of the U.S. population has some level of difficulty swallowing these conventional dosage forms. Pediatric and geriatric patients are particularly susceptible to swallowing difficulties, including dysphagia. An alternative to these conventional dosage forms is the use of solid dosage forms that rapidly dissolve or disintegrate in the oral cavity, commonly called orally disintegrating tablets (hereinafter ODT.)

ODT can be taken without chewing or the need for water, thereby providing ease of administration and improving patient compliance. ODT are particularly beneficial for meeting the needs of pediatric and geriatric patients, as well as patients with dysphagia.

A number of commercial ODT products employing various manufacturing technologies are available. Illustrative examples include ZYDIS® by Cardinal Health, prepared by a freeze drying method; FlashDose® by Biovail, prepared by “cotton candy spinning” and compression; AdvaTab® by Eurand, prepared by direct compression using non-effervescent excipients; and OraSolv® or DuraSolv®, both by Cima, prepared by direct compression including effervescent ingredients in the formulation.

There are disadvantages, including reduced product quality as well as processing difficulties, associated with products currently commercially available. For example, the process of freeze drying the tablet formulation requires water to be removed by sublimation in the product preparation process. This method creates an amorphous porous structure that allows rapid disintegration of the ODT. Unfortunately, conventional freeze drying methods are usually very time-consuming, often require specialized manufacturing equipment and are limited to low doses of active pharmaceutical ingredients (hereinafter API), typically less than 50 mg. The mechanical strength of the resulting tablets is usually so poor that the tablets require specialized blister packaging to protect tablet integrity.

Molding is another conventional method that has been used to produce ODT. The process requires the use of heat and solvents including water. The molded ODT provides a fast disintegration time in the oral cavity because of the porous structure of the product matrix as well as the use of water soluble materials to form the matrix. The mechanical strength of molded ODT is typically weak, the production cost is high and the process is often complicated.

ODT can also be produced by a direct compression method with the inclusion of an effervescent material such as sodium bicarbonate and/or citric acid in the tablet formulation. When the effervescent tablets are exposed to moisture a chemical reaction takes place wherein the effervescent materials react with water, yielding carbon dioxide as a byproduct, resulting in tablet disintegration. For this reason, ODT which include effervescent materials in the formulation are so highly sensitive to moisture that the ODT require a specialized packaging method to avoid moisture penetration during storage. They also typically exhibit an unpleasant mouth-feel and slow oral disintegration time.

Another method to produce ODT is to employ a direct compression method under lower compression force. The formulation usually includes various combinations of sugars, super-disintegrants, starches, cellulose derivatives and water-insoluble inorganic salts. The typical oral disintegration time is greater than 40 seconds. The ODT produced by this process exhibit a high degree of friability and produce a chalky taste and dry mouth-feel when placed in the mouth. A further disadvantage is that these ODT have such poor mechanical strength that the ODT tend to crumble and break prior to administration. This leads to uncertainty as to the amount of API actually dosed to the patient.

There is therefore a need for improved ODT formulations and methods of manufacture.

### SUMMARY OF INVENTION

An illustrative aspect of the present invention is to provide an improved orally dissolving tablet. The improved ODT comprises at least one water-insoluble hydrophobic inorganic salt, wherein the water-insoluble hydrophobic inorganic salt(s) absorbs no more than about 0.2% water by weight at relative humidity of 95% at 25° C., in combination with at least one water-insoluble inorganic salt, wherein the water-insoluble inorganic salt(s) absorbs between about 0.3% and about 3.0% water by weight at a relative humidity of 95% at 25° C., and at least one active pharmaceutical ingredient.

In another illustrative aspect of the present invention, there is provided an ODT composition comprising about 18% to about 88% by weight of at least one water-soluble excipient; about 2% to about 20% by weight of at least one water swellable polymeric material; about 3% to about 25% by weight of at least one water-insoluble hydrophobic inorganic salt that absorbs no more than about 0.2% water by weight at a relative humidity of 95% at 25° C.; about 3% to about 25% by weight of at least one water-insoluble inorganic salt that absorbs between about 0.3% and about 3.0% water by weight at a relative humidity of 95% at 25° C.; and at least one active pharmaceutical ingredient. The particle size of the water swellable polymeric material(s) and the water-insoluble inorganic salt(s) and the water-insoluble hydrophobic inorganic salt(s) is typically not more than about 80 μm by Malvern particle size analysis.

In yet another aspect of the present invention there is provided a method of making orally disintegrating granules. The method comprises granulating a mixture including that includes at least one water-soluble excipient, at least one water swellable polymeric material, at least one water-insoluble hydrophobic inorganic salt that absorbs no more than about 0.2% water by weight at a relative humidity of 95% at 25° C., and at least one water-insoluble inorganic salt that absorbs between about 0.3% and about 3.0% water by weight at a relative humidity of 95% at 25° C. with water to form wet granules. The wet granules are dried to form substantially dry granules, and the substantially dry granules are milled to produce orally disintegrating granules of a desired size.

In yet a further aspect of the present invention there is provided a method of making orally disintegrating granules. The improved method comprises granulating a mixture including about 18% to about 90% by weight of at least one water-soluble excipient; about 2% to about 20% by weight of at least one water swellable polymeric material; about 3% to about 25% by weight of at least one water-insoluble hydrophobic inorganic salt that absorbs no more than about 0.2% water by weight at a relative humidity of 95% at 25° C., and about 3% to about 25% by weight of at least one water-insoluble inorganic salt that absorbs between about 0.3% and about 3.0% water by weight at a relative humidity of 95% at 25° C. with water to form wet granules. The wet granules are then substantially dried and milled to a desired size.

In still another aspect of the present invention there is provided a method of making a rapidly disintegrating tablet. The method comprises granulating a mixture including about 18% to about 98% by weight of at least one water-soluble excipient, about 2% to about 20% by weight of at least one water swellable polymeric material, about 3% to about 25% by weight of at least one water-insoluble hydrophobic inorganic salt that absorbs no more than about 0.2% water by weight at a relative humidity of 95% at 25° C., and about 3% to about 25% by weight of at least one water-insoluble inorganic salt that absorbs between about 0.3% and about 3.0% water by weight at a relative humidity of 95% at 25° C. with water to form wet granules. The wet granules are then substantially dried and milled to a desired size. The granules are then compressed into a tablet.

### DETAILED DESCRIPTION

There is provided in accordance with the present invention, an improved ODT composition which overcomes the disadvantages of the prior art methods described herein. There is further provided an improved method of manufacturing ODT which uses commonly available manufacturing equipment for granulation, blending and tableting. The improved ODT disclosed herein are prepared by direct compression of a mixture of pharmaceutical excipients comprised of a) at least one water-soluble excipient; b) at least one water-swellable polymeric material including a disintegrant; c) at least one water-insoluble hydrophobic inorganic salt; and d) at least one water-insoluble inorganic salt with less hydrophobicity compared to component c). These components may be formed into granules, and may include other commonly used excipients. In an illustrative embodiment the tablets comprising these components are formed into tablets by direct compression, optionally using a lubricant. The fast disintegrating tablets prepared using these components exhibit desirable performance properties such as sufficient hardness, low friability, quick disintegration time and good mouth-feel, when compared to conventional ODT. Further, the improved hardness and low friability make the improved ODT suitable for packaging in conventional bottles and push through blister packs using conventional equipment for storage, transportation and commercial distribution.

In an illustrative embodiment of the present invention, the improved ODT comprises about 18% to about 88% by weight of the water-soluble excipient(s); about 2% to about 20% by weight of the water-swellable polymeric material(s); about 3% to about 25% by weight of water-insoluble hydrophobic inorganic salt(s); and about 3% to about 25% by weight of at least one water-insoluble inorganic salt(s).

In an illustrative embodiment, the ratio of the water-insoluble hydrophobic inorganic salt(s) to the water-insoluble inorganic salt(s) is from about 1:10 to about 10:1.

As used herein, the term “water soluble excipient” refers to a solid material or a mixture of materials that readily dissolve in water. Suitable water soluble excipients include sugars, for example sucrose, maltose, lactose, glucose, mannose and mixtures thereof, and sugar alcohols, for example mannitol, erythritol, sorbitol, xylitol, lactitol, maltitol and mixtures thereof. The preferred water-soluble excipients are non-hygroscopic, or have a low degree of hygroscopicity, typically absorbing water only above relative humidity of 95% at 20° C. Presently preferred water soluble excipients include spray dried mannitol and/or erythritol.

The term “water-swellable polymeric material” refers to a disintegrant that takes up water and swells rapidly in contact with water, or when administered to a patient in less than 2 ml saliva. Suitable disintegrants include modified starches, sodium starch glycolate, croscarmellose sodium, crospovidone, low substituted hydroxypropyl cellulose and mixtures thereof. Among the disintegrants, polyplasdone XL-10 (ISP Technologies Inc.) a cross-linked homopolymer of N-vinyl-2-pyrrolidone having porous particle morphology and a particle size of not more than about 90 μm, with a mean particle size of not more than about 30 μm by Malvern particle size analysis.

The term “water-insoluble hydrophobic inorganic salt” refers to an inorganic solid in powder form that absorbs no more than about 0.2% by weight water at relative humidity of 95% at 25° C., and typically has a particle size of about 50 μm or less. Suitable water-insoluble inorganic salts include calcium diphosphate (dihydrate) and calcium triphosphate, both anhydrous and hydrate forms, having particle size of less than about 40 μm, Talc Imperial USP BC (MPSI) having particle size smaller than about 50 μm and a mean particle size of less than about 15 μm by Malvern particle size analysis, and Talc Lo-Micron USP BC (MPSI) having particle size smaller than about 40 μm and a mean particle size of about 1.2 μm by sieve analysis.

The term “water-insoluble inorganic salt” refers to an inorganic solid in powder form that absorbs between about 0.3% and about 3.0% by weight water at relative humidity of 95% at 25° C., and typically has a mean particle size of about 80 μm or less. Suitable water-insoluble inorganic salts include hydrophobically modified calcium silicate such as RxCipients FM1000 by HUBER Engineered Materials having particle size of about 40 μm or less by sieve analysis and Talc USP BC 300 (MPSI) having particle size smaller than about 80 μm and a mean particle size of not more than about 15 μm by Malvern particle size analysis.

The granules and subsequent tablets may include an API. Suitable API include, but are not limited to non-steroidal anti-inflammatory agents, contraceptives, opioids, thyroid and antithyroid drugs, gout therapy drugs, cough and cold drugs, anticonvulsants, antirheumatic drugs, anti-migraine drugs, anti-parasite, hormonal drugs, mitotic inhibitors, immunosuppressants, antihypersensitive drugs, calcium-channel blocking agents, antidepressants, anxiolytics, neurodegenerative disease drugs, bismuth salts, coagulants, antiulcer agents, coronary vasodilators, peripheral vasodilators, oral antibacterial and antifungal agents, antispasmodics, antitussive agents, antiasthmatic agents, bronchodilators, diuretics, muscle relaxants, brain metabolism altering drugs, tranquilizers, beta blockers, antiarrhythmic agents, anticoagulants, antiepileptic agents, antiemetics, hypo- and hypertensive drugs, sympathomimetic agents, expectorants, oral antidiabetic agents, circulatory agents, nutritional supplements, pollakiuria remedies, angiotension-converting enzyme inhibitors, antiviral agents, antihistamines nasal decongestants and mixtures thereof.

Another illustrative embodiment of the invention relates to a method for producing rapidly dissolving granules. The improved method comprises granulating of a mixture of the improved ODT formulations described herein. The mixture of the four primary components noted above can be granulated by adding enough water to provide sufficient granule strength during the subsequent drying process, typically about 10% to about 45% by weight water to the improved ODT formulation and using a low shear granulator, a high shear granulator or a fluid bed granulator to make granules from the dry materials. The resulting wet mass is then substantially dried, for example in a fluid bed chamber or a drying oven, until about 0.5% to about 4.0% water by weight for good flow. The resulting dry granules are milled to produce the desired particle size distribution, yielding rapidly disintegrating granules. The granules typically have a particle size of less than about 700 μm. In an illustrative, non-limiting embodiment of the present invention, the mean particle size is from about 100 μm to about 200 μm.

In yet another illustrative embodiment of the invention, a method for preparing ODT is provided. The method comprises blending the granules of the present invention with at least one optional lubricant and then compressing the resulting mixture to form a tablet. At least one API may be added, either prior to granulation or prior to compression of the granules into a tablet.

Alternatively, additives other than the four primary components described in this invention, including but not limited to colorants, flavorings, lubricants, sweeteners, water soluble polymers, silicified microcrystalline cellulose and mixtures thereof may be added to the formulation prior to or after granulation, if desired. The improved ODT prepared by the methods described herein provide a rapid disintegration time less than 30 seconds, often less than 25 seconds in the mouth, and exhibit a smooth mouth-feel. The improved ODT have a low degree of friability less than 0.8% by weight, and a hardness of greater than 4 kP, so that the tablets are suitable for packaging in conventional HDPE bottles or push through blister packages. Another benefit is that the present methods of granulation and tablet preparation can be accomplished using conventional manufacturing equipment such as V-blender, low or high shear granulator, fluid-bed dryer, roller compactor and tablet press.

### EXAMPLES

### Example 1

### Fast Disintegrating Tablets From Dry Blend

A powder mixture of 600 g of spray dried mannitol (Pearlitol 200SD, Roquette), 100 g of crospovidone XL-10 (SPI Pharma), 160 g of hydrophobically modified calcium silicate (RxCipients FM 1,000, Huber engineered material), and 130 g of Talc Imperial (MPSI) was blended in a 2-quart-V blender (Twin shell) for 30 minutes and then was lubricated with 10 g of magnesium stearate dihydrate (98+ purity, Mallinckrodt, Inc.) for 5 minutes. The lubricated blend was compressed into tablets at a main compression force of 15 kilonewtons and a precompression force of 1,000 newtons at 60 rpm in a 0.4062 inch die with a flat faced and beveled edge punch by a 16-station Manesty Beta press. The approximate weight of each tablet was 400 mg. The physical properties of the tablets were evaluated as follows:

(A) Hardness Test:

The ODT tablet crushing load, which is the force (Kilopond, Kp) required to break a tablet into halves by compression in the diametral direction, was measured with a hardness tester (Varian Hardness tester, VK-200).

(B) Friability Test:

The tablet friability test method was performed by a Varian Friabilator according to the USP 25 tablet friability method described in <1216> Tablet Friability of the General chapters describing General Test and Assays.

(C) In Vitro Disintegration Test:

One Whatman filter disc (21 mm in diameter) was placed in each well of a Corning 12-well polystyrene microplate (22 mm in diameter). One milliliter of 0.1% Sensient blue #1 dye solution (similar to the in vivo condition) was then added into each well. An ODT tablet was carefully placed on the surface of the wet filter paper disc in each well using a pair of forceps. Finally, the total wetting time was recorded as the time required for the blue dye solution to cover the top surface of the tablet as the in vitro disintegration time.

Evaluation of In Vivo Disintegration Test:

In vivo test of ODT containing no API were conducted on volunteers. Volunteers were randomized to receive the treatments and then directed to clean their mouths with water. ODT tablets were placed on their tongue, and a stopwatch was started immediately as the ODT contacted the surface of the tongue. The participants were allowed to move the ODTs against the upper roof of the mouth with their tongues and to cause a gentle tumbling action on the tablet without biting on it or tumbling it from side to side. Immediately after the tablet disintegrated into small particles, the stopwatch was stopped and the in vivo disintegration time recorded.

Results: The average in vitro and in vivo disintegration times were 25 seconds and 23 seconds, respectively. The average hardness of the tablets was 5.0 Kp. The average friability of the tablets was 0%.

### Example 2

### Fast Disintegrating Tablets From Dry Blend

A powder mixture of 630 g of spray dried mannitol (Pearlitol 200SD, Roquette), 130 g of crospovidone XL-10 (SPI Pharma), 160 g of Talc USP BC 300 (MPSI), and 70 g of Talc Imperial (MPSI) was blended in a 2-quart-V blender (Twin shell) for 30 minutes and then was lubricated with 10 g of magnesium stearate dihydrate (98+ purity, Mallinckrodt Inc.) for 5 minutes. The lubricated blend was compressed into tablets at a main compression force of 15 kilonewtons and a precompression force of 1,000 newtons at 60 rpm in a 0.4062 inch die with flat faced and beveled edge punches by a 16-station Manesty Beta press. The weight of each tablet was 300 mg. The physical properties of the tablets were evaluated according to the procedures described in Example 1. The average in vitro and in vivo disintegration times were 19.3 seconds and 20 seconds, respectively. The average hardness of the tablets was 6.3 kP.

### Example 3

### Fast Disintegrating Tablets From Low Shear Wet Granulation

A powder mixture of 600 g of spray dried mannitol (Pearlitol 200SD, Roquette), 100 g of crospovidone XL-10 (SPI Pharma), 160 g of hydrophobically modified calcium silicate (RxCipients FM 1,000, Huber chemical), and 130 g of Talc Imperial (MPSI) was blended in a 2-quart-V blender (Twin shell) for 30 minutes. 700 g of the dry blend was transferred into a 4½ quart KitchenAid classic stand mixer. 150 g of purified water was sprayed over 13 minutes while mixing with a wire whisk attached to the mixer at speed control of 4. The obtained wet mass was then passed through a No. 6 sieve. The screened wet mass was transferred onto an aluminum tray for drying. The wet granules were dried in a 50° C. dry oven (Scientific Products DX-31) for 8 hours. The dried granules (water content between 0.5 and 4.0% (w/w)) were passed through a No. 20 sieve. 990 g of the granule was lubricated with 10 g of magnesium stearate dihydrate (98+ purity, Mallinckrodt, Inc.) in a 4-quart-V blender (Twin shell) for 5 minutes. The lubricated blend was compressed into tablets at a main compression force of 15 kilonewtons and a precompression force of 1,000 newtons at 60 rpm in a 0.4062 inch die with flat faced and beveled edge punches by a 16-station Manesty Beta press. The approximate weight of each tablet was 300 mg. The physical properties of the tablets were evaluated according to the procedures described in Example 1. The average in vitro and in vivo disintegration times were 29 seconds and 27 seconds, respectively. The average hardness of the tablets was 6.3 kP. The average friability of the tablets was 0%.

### Example 4

### Fast Disintegrating Tablets From Low Shear Wet Granulation

A powder mixture of 630 g of spray dried mannitol (Pearlitol 200SD, Roquette), 130 g of crospovidone XL-10 (SPI Pharma), 160 g of Talc USP BC 300, and 70 g of Talc Imperial (MPSI) was blended in a 2-quart-V blender (Twin shell) for 30 minutes. 700 g of the dry blend was transferred into a 4½ quart KitchenAid classic stand mixer. 120 g of purified water was sprayed over 10 minutes while mixing with a wire whisk attached to the mixer at speed control of 4. The obtained wet mass was then passed through a No. 6 sieve. The screened wet mass was transferred onto an aluminum tray for drying. The wet granules were dried in a 50′C dry oven (Scientific Products DX-31)for 8 hours. The dried granules (water content between 0.5 and 4.0% (w/w)) were passed through a No. 20 sieve. 990 g of the granules were lubricated with 10 g of magnesium stearate dihydrate (98+ purity, Mallinckrodt Inc.) in a 4-quart-V blender (Twin shell) for 5 minutes. The lubricated blend was compressed into tablets at a main compression force of 12 kilonewtons and a precompression force of 1,000 newtons at 60 rpm in a 0.4062 inch die with flat faced and beveled edge punches by a 16-station Manesty Beta press. The approximate weight of each tablet was 300 mg. The physical properties of the tablets were evaluated according to the procedures described in Example 1. The average in vitro and in vivo disintegration times were 28 seconds and 24 seconds, respectively. The average hardness of the tablets was 5.1 kP. The average friability of the tablets was 0%.

### Example 5

### Fast Disintegrating Tablets From High Shear Wet Granulation

A powder mixture of 600 g of spray dried mannitol (Pearlitol 200SD, Roquette), 100 g of crospovidone XL-10 (SPI Pharma), 160 g of hydrophobically modified calcium silicate (RxCipients FM 1,000, Huber chemical), and 130 g of Talc Imperial (MPSI) was blended in a 2-quart-V blender (Twin shell) for 30 minutes. 900 g of the dry blend was transferred into a 5 liter high shear mixing bowl. A Glatt B60 Vertical granulator was used for mixing at an impeller speed of 20 rpm and chopper speed of 1,000 rpm for two minutes. Then 306 ml of purified water was pumped at a rate of 15 ml/min. at an impeller speed of 200 rpm and chopper speed of 1,500 rpm. The wet mass was mixed for 2 more minutes after stopping water addition. The wet mass was sieved through a No. 6 sieve and dried either in a dry oven or a fluid bed dryer or on a tray to air dry. The dried granules (water content between 0.5 and 4.0% (w/w)) were passed through a No. 20 sieve. 990 g of the granules were lubricated with 10 g of magnesium stearate dihydrate (98+ purity, Mallinckrodt Inc.) in a 4-quart-V blender (Twin shell) for 5 minutes. The lubricated blend was compressed into tablets at a main compression force of 15 kilonewtons and a precompression force of 1,000 newtons at 60 rpm in a 0.4062 inch die with flat faced and beveled edge punches by a 16-station Manesty Beta press. The approximate weight of each tablet was 400 mg. The physical properties of the tablets were evaluated according to the procedures described in Example 1. The average in vitro disintegration time was 19 seconds. The average hardness of the tablets was 9.1 kP. The average friability of the tablets was 0%.

### Example 6

### Fast Disintegrating Tablets From High Shear Wet Granulation

A powder mixture of 630 g of spray dried mannitol (Pearlitol 200SD, Roquette), 130 g of crospovidone XL-10 (SPI Pharma), 160 g of Talc USP BC 300, and 70 g of Talc Imperial (MPSI) was blended in a 2-quart-V blender (Twin shell) for 30 minutes. 900 g of the dry blend was transferred into a 5 liter high shear mixing bowl. A Glatt B60 Vertical granulator was used for mixing at an impeller speed of 20 rpm and chopper speed of 1,000 rpm for two minutes. Then, 288 ml of purified water was pumped at a rate of 14 ml/min. at an impeller speed of 200 rpm and chopper speed of 1,500 rpm. The wet mass was mixed for 2 more minutes after stopping water addition. The wet mass was sieved through a No. 6 sieve and dried either in a dry oven or a fluid bed dryer or on a tray to air dry. The dried granules (water content between 0.5 and 4.0% (w/w)) were passed through a No. 20 sieve. 990 g of the granules were lubricated with 10 g of sodium stearyl fumarate (SPI Pharma) in a 4-quart-V blender (Twin shell) for 5 minutes. The lubricated blend was compressed into tablets at a main compression force of 15 kilonewtons and a precompression force of 1,000 newtons at 60 rpm in a 0.4062 inch die with flat faced and beveled edge punches by a 16-station Manesty Beta press. The approximate weight of each tablet was 400 mg. The physical properties of the tablets were evaluated according to the procedures described in Example 1. The average in vitro disintegration time was 18 seconds. The average hardness of the tablets was 9.5 kP. The average friability of the tablets was 0%.

### Example 7

### Fast Disintegrating Tablets From a Combination of the Fast Dissolving Granules and PROSOLV HD 90

A powder mixture of 3.0 g of the granules from Example 3, 0.50 g of crospovidone XL-10, 0.50 g of PROSOLV HD90 (silicified microcrystalline cellulose, JRS Pharma), and 5.90 g of spray dried mannitol (Pearlitol 200SD, Roquette) was hand blended in a 20 ml glass vial for 3 minutes and then lubricated for 30 seconds with 0.1 g of sodium stearyl fumarate (Lubripharm). The lubricated blend was compressed into tablets at 2,000 lb in a 0.362 inch die by a Natoli Carver press. The physical properties of the tablets were evaluated according to the procedures described in Example 1. The approximate weight of the tablet was 300 mg. The average disintegration time in the mouth was 18 seconds. The average hardness of the tablets was 6.0 Kp.

### Example 8

### Fast disintegrating tablets of 8 mg Chlorpheniramine (Taste Masked Chlorpheniramine Resinate)

A dry blend of 600 g of spray dried mannitol (Pearlitol 200SD, Roquette), 100 g of crospovidone XL-10 (SPI Pharma), 100 g of hydrophobically modified calcium silicate (RxCipients FM 1,000, Huber engineered material), 200 g of Talc Imperial (MPSI) was prepared in a 2-quart V-blender (Twin shell) for 30 minutes. 700 g of the dry blend was transferred into a 4 and ½ quart Kitchen Aid classic stand mixer. 150 g of purified water was sprayed over 13 minutes while mixing with a wire whisk attached to the mixer at speed control of 4. The obtained wet mass was then passed through a No. 6 sieve. The screened wet mass was transferred onto an aluminum tray for drying. The wet granules were dried in a 50° C. dry oven (Scientific Products DX-31) for 8 hours. The dried granules (water content between 0.5 and 4.0% (w/w)) were passed through a No. 20 sieve. 0.846 g of chlorpheniramine polistirex (Purolite irregular type of ion exchange resin containing 32.78% (w/w) chlorpheniramine on dry basis, 3.88% water content), 3.0 g of the granules, 4.554 g of spray dried mannitol (Pearlitol 200SD, Roquette), 1.0 g of PROSOLV HD 90 (silicified microcrystalline cellulose, JRS Pharma), 0.5 g of Crospovidone XL-10, and 0.1 g of sodium stearyl fumarate (Lubripharm, SPI Pharma) were hand mixed in a 20 ml glass vial for 3 minutes. The blend was compressed into tablets at 2,000 lb in a 0.362 inch die by a Natoli Carver press. The approximate weight of the tablet was 310 mg. The average disintegration time in the USP method was 11 seconds.

### Example 9

### Fast Disintegrating Tablets of 10mg Hydrocodone (Taste Masked Hydrocodone Resinate)

A dry blend of 600 g of spray dried mannitol (Pearlitol 200SD, Roquette), 100 g of crospovidone XL-10 (SPI Pharma), 100 g of hydrophobically modified calcium silicate (RxCipients FM 1,000, Huber engineered material), and 200 g of Talc Imperial (MPSI) was prepared in a 2-quart V-blender (Twin shell) for 30 minutes. 700 g of the dry blend was transferred into a 4½ quart KitchenAid classic stand mixer. 150 g of purified water was sprayed over 13 minutes while mixing with a wire whisk attached to the mixer at speed control of 4. The obtained wet mass was then passed through a No. 6 sieve. The screened wet mass was transferred onto an aluminum tray for drying. The wet granules were dried in a 50° C. dry oven (Scientific Products DX-31) for 8 hours. The dried granules (water content between 0.5 and 4.0% (w/w)) went through a No. 20 sieve. 1.62 g of hydrocodone polistirex (Dow spherical type of ion exchange resin containing 22.16% (w/w) hydrocodone on dry basis, 7.33% water content), 3.0 g of the granule, 3.78 g of spray dried mannitol (Pearlitol 200SD, Roquette), 1.0 g of PROSOLV HD 90 (silicified microcrystalline cellulose, JRS Pharma), 0.5 g of Crospovidone XL-10, and 0.1 g of sodium stearyl fumarate (Lubripharm, SPI Pharma) were hand mixed in a 20 ml glass vial for 3 minutes. The blend was compressed into tablets at 2,000 lb in a 0.362 inch die by a Natoli Carver press. The approximate weight of the tablet was 306 mg. The average disintegration time in the USP method was 9 seconds.

### Example 10

### Fast Disintegrating Tablets Comprised of a Combination of 10 mg Hydrocodone and 8 mg Chlorpheniramine (Taste Masked Hydrocodone and Chlorpheniramine Resinates)

A dry blend of 600 g of spray dried mannitol (Pearlitol 200SD, Roquette), 100 g of crospovidone XL-10 (SPI Pharma), 100 g of hydrophobically modified calcium silicate (RxCipients FM 1,000, Huber engineered material), and 200 g of Talc Imperial (MPSI) was prepared in a 2-quart V-blender (Twin shell) for 30 minutes. 700 g of the dry blend was transferred into a 4½ quart KitchenAid classic stand mixer. 150 g of purified water was sprayed over 13 minutes while mixing with a wire whisk attached to the mixer at speed control of 4. The obtained wet mass was then passed through a No. 6 sieve. The screened wet mass was transferred onto an aluminum tray for drying. The wet granules were dried in a 50° C. dry oven (Scientific Products DX-31) for 8 hours. The dried granules (water content between 0.5 and 4.0% (w/w)) were passed through a No. 20 sieve. 1.62 g of hydrocodone polistirex (Dow spherical type of ion exchange resin containing 22.16% (w/w) hydrocodone on dry basis, 7.33% water content), 0.846 g of chlorpheniramine polistirex (Purolite irregular type of ion exchange resin containing 32.78% (w/w) chlorpheniramine on dry basis, 3.88% water content), 3.0 g of the granules, 2.934 g of spray dried mannitol (Pearlitol 200SD, Roquette), 1.0 g of PROSOLV HD 90 (silicified microcrystalline cellulose, JRS Pharma), 0.5 g of Crospovidone XL-10, and 0.1 g of sodium stearyl fumarate (Lubripharm, SPI Pharma) were hand mixed in a 20 ml glass vial for 3 minutes. The blend was compressed into tablets at 2,000 lb in a 0.362 inch die by a Natoli Carver press. The approximate weight of the tablets was 307 mg. The average disintegration time in the USP method was 8 seconds. Having described the invention in detail, those skilled in the art will appreciate that modifications may be made to the invention without departing from its spirit and scope. Therefore, it is not intended that the scope of the invention be limited to the specific embodiments described. Rather, it is intended that the appended claims and their equivalents determine the scope of the invention.

What is claimed is:

1. An orally dissolving tablet comprising:
a) at least one water-insoluble hydrophobic inorganic salt, wherein the water-insoluble hydrophobic inorganic salt absorbs not more than 0.2% by weight water at a relative humidity of 95% at 25° C. in combination with at least one water-insoluble inorganic salt wherein the water-insoluble inorganic salt absorbs between 0.3% and 3.0% by weight water at a relative humidity of 95% at 25° C.; and
b) at least one active pharmaceutical ingredient.

2. The tablet of claim 1 comprising:
a) 3% to 25% by weight of the at least one water-insoluble hydrophobic inorganic salt; and
b) 3% to 25% by weight of the at least one water-insoluble inorganic salt.

3. The tablet of claim 2 wherein the ratio of the at least one water-insoluble hydrophobic inorganic salt to the at least one water-insoluble inorganic salt is from about 1:10 to about 10:1.

4. The tablet of claim 1 further comprising:
a) at least one water-soluble excipient selected from the group consisting of sugar, sugar alcohols and mixtures thereof;
b) at least one water-swellable polymeric material including a disintegrant; and
wherein,
the at least one water-insoluble hydrophobic inorganic salt is selected from the group consisting of calcium diphosphate, calcium triphosphate, talc with a particle size less than 50 μm, and mixtures thereof; and
the at least one water-insoluble inorganic salt is selected from the group consisting of modified calcium silicate, talc with a particle size smaller than 80 μm and a mean particle size of 15 μm, and mixtures thereof.

5. The tablet of claim 4 wherein the disintegrant includes at least one modified starch selected from the group consisting of sodium starch glycolate, croscarmellose sodium, crospovidone, low substituted hydroxypropyl cellulose and mixtures thereof; and
the at least one water-soluble excipient is selected from the group consisting of sucrose, maltose, lactose, glucose, mannose, mannitol, sorbitol, xylitol, erythritol, lactitol, maltitol and mixtures thereof.

6. The tablet of claim 1 further comprising:
a) 18% to 88% by weight of at least one water-soluble excipient;
b) 2% to 20% by weight of at least one water-swellable polymeric material;
c) 3% to 25% by weight of the at least one water-insoluble hydrophobic inorganic salt; and
d) 3% to 25% by weight of the at least one water-insoluble inorganic salt.

7. The tablet of claim 1 wherein the at least one water-insoluble hydrophobic inorganic salt and the at least one water-insoluble inorganic salt have a particle size of no more than 80 μm.

8. The tablet of claim 1 further comprising at least one additive selected from the group consisting of colorants, sweeteners, flavorants, binders, lubricants and mixtures thereof.

9. The tablet of claim 1 wherein the at least one active pharmaceutical ingredient is selected from the group consisting of non-steroidal anti-inflammatory agents, contraceptives, opioids, thyroid and antithyroid drugs, gout therapy drugs, cough and cold drugs, anticonvulsants, antirheumatic drugs, anti-migraine drugs, anti-parasite, hormonal drugs, mitotic inhibitors, immunosuppressants, antihypersensitive agents, calcium-channel blocking agents, antidepressants, anxiolytics, neurodegenerative disease drugs, bismuth salts, coagulants, antiulcer agents, coronary vasodilators, peripheral vasodilators, oral antibacterial and antifungal agents, antispasmodics, antitussive agents, antiasthmatic agents, bronchodilators, diuretics, muscle relaxants, brain metabolism altering drugs, tranquilizers, beta blockers, antiarrhythmic agents, anticoagulants, antiepileptic agents, antiemetics, hypo- and hypertensive agents, sympathomimetic agents, expectorants, oral antidiabetic agents, circulatory agents, nutritional supplements, pollakiuria remedies, angiotension-converting enzyme inhibitors, antiviral agents, antihistamines, and nasal decongestants.

10. An orally dissolving tablet composition comprising:
a) 18% to 88% by weight of at least one water-soluble excipient;
b) 2% to 20% by weight of at least one water swellable polymeric material;
c) 3% to 25% by weight of at least one water-insoluble hydrophobic inorganic salt that absorbs no more than 0.2% by weight water at a relative humidity of 95% at 25° C.;
d) 3% to 25% by weight of at least one water-insoluble inorganic salt that absorbs between 0.3% and 3.0% by weight water at a relative humidity of 95% at 25° C.; and
e) at least one active pharmaceutical ingredient, wherein the particle size of the water-insoluble hydrophobic inorganic salt and the water-insoluble inorganic salt is no more than 80 μm.

11. The tablet of claim 10 wherein the ratio of the at least one water-insoluble hydrophobic inorganic salt to the at least one water-insoluble inorganic salt is from about 1:10 to about 10:1.

12. The tablet of claim 10 wherein:
a) the at least one water-soluble excipient is selected from the group consisting of sugar, sugar alcohols and mixtures thereof;
b) the at least one water-swellable polymeric material includes at least one disintegrant;
c) the at least one water-insoluble hydrophobic inorganic salt is selected from the group consisting of calcium diphosphate, calcium triphosphate, talc having a particle size less than 50 μm and mixtures thereof; and
d) the at least one water-insoluble inorganic salt is selected from the group consisting of physically modified calcium silicate and a talc having particle size smaller than 80 μm and a mean particle size of 15 μm and mixtures thereof.

13. The tablet of claim 10 wherein the disintegrant is selected from the group consisting of modified sodium starches, croscarmellose sodium, crospovidone, low substituted hydroxypropyl cellulose and mixtures thereof; and
the at least one water-soluble excipient is selected from the group consisting of sucrose, maltose, lactose, glucose, mannose, mannitol, sorbitol, xylitol, erythritol, lactitol, maltitol and mixtures thereof.

14. An orally dissolving granule comprising:
a) 18% to 88% by weight of at least one water-soluble excipient;
b) 2% to 20% by weight of at least one water-swellable polymeric material;
c) 3% to 25% by weight of at least one water-insoluble hydrophobic inorganic salt, wherein the water-insoluble hydrophobic inorganic salt absorbs no more than 0.2% water by weight at relative humidity of 95% at 25° C.; and
d) 3% to 25% by weight of at least one water-insoluble inorganic salt, wherein the water-insoluble inorganic salt absorbs between 0.3% and 3.0% water by weight at a relative humidity of 95% at 25° C.

15. The granule of claim 14 wherein the at least one water-insoluble hydrophobic inorganic salt and the at least one water-insoluble inorganic salt have a particle size of no more than about 80 μm.

16. The granule of claim 14 further including at least one active pharmaceutical ingredient.

17. The granule of claim 14 wherein:
a) the at least one water-soluble excipient is selected from the group consisting of sugar, sugar alcohols and mixtures thereof;
b) the at least one water-swellable polymeric material includes at least one disintegrant;
c) the at least one water-insoluble hydrophobic inorganic salt is selected from the group consisting of calcium diphosphate, calcium triphosphate, talc with a particle size less than about 50 μm, and mixtures thereof; and
d) the at least one water-insoluble inorganic salt is selected from the group consisting of modified calcium silicate and talc with a particle size smaller than about 80 μm and a mean particle size of about 15 μm and mixtures thereof.

18. The granule of claim 17 wherein the at least one selected disintegrant is selected from the group consisting of modified starch, croscarmellose sodium, crospovidone, low substituted hydroxypropyl cellulose and mixtures thereof; and the water-soluble excipient is selected from the group consisting of sucrose, maltose, lactose, glucose, mannose, mannitol, sorbitol, xylitol, erythritol, lactitol, maltitol, and mixtures thereof.

19. The granule of claim 14 further comprising at least one additive selected from the group consisting of at least one colorant, at least one sweetener, at least one flavorant, at least one binder, at least one lubricant, and mixtures thereof.

20. The granule of claim 14 further comprising an active pharmaceutical ingredient.

21. A method of making orally dissolving granules, the method comprising
a) granulating a mixture including;
i) at least one water-soluble excipient;
ii) at least one water swellable polymeric material;
iii) at least one water-insoluble hydrophobic inorganic salt that absorbs no more than 0.2% water by weight at a relative humidity of 95% at 25° C.; and
iv) at least one water-insoluble inorganic salt that absorbs between 0.3% and 3.0% water by weight at a relative humidity of 95% at 25° C.;
with water to form wet granules;
b) drying the wet granules to form substantially dry granules; and
c) milling the substantially dry granules to produce orally dissolving granules of a desired size.

22. The method of claim 21 wherein the particle size of the orally dissolving granules is no more than about 700 μm.

23. The method of claim 21 further comprising adding a lubricant to the substantially dried granules.

24. The method of claim 21 further comprising adding at least one active pharmaceutical ingredient to the substantially dried granules.

25. The method of claim 21 wherein the mixture includes:
a) 18% to 98% by weight of the at least one water-soluble excipient;
b) 2% to 20% by weight of the at least one water-swellable-polymeric material;
c) 3% to 25% by weight of the at least one water-insoluble hydrophobic inorganic salt that absorbs no more than 0.2% water by weight at a relative humidity of 95% at 25° C.; and
d) 3% to 25% by weight of the at least one water-insoluble inorganic salt that absorbs between 0.3% and 3.0% water by weight at a relative humidity of 95% at 25° C.

26. A method of making orally dissolving granules, the method comprising
a) granulating a mixture including:
i) 18% to 90% by weight of at least one water-soluble excipient;
ii) 2% to 20% by weight of at least one water swellable polymeric material;
iii) 3% to 25% by weight of at least one water-insoluble hydrophobic inorganic salt that absorbs no more than 0.2% water by weight at a relative humidity of 95% at 25° C.; and
iv) 3% to 25% by weight at least one water-insoluble inorganic salt that absorbs between 0.3% and 3.0% water by weight at a relative humidity of 95% at 25° C.
with water to form wet granules;
b) drying the wet granules to form substantially dry granules; and
c) milling the substantially dry granules to produce orally dissolving granules of a desired size.

27. The method of claim 26 wherein the ratio of the at least one water-insoluble hydrophobic inorganic salt to the at least one water-insoluble inorganic salt is from about 1:10 to about 10:1.

28. The method of claim 26 wherein the particle size of the orally dissolving granules is no more than about 700 μm.

29. A method of making a rapidly dissolving tablet, the method comprising:
a) granulating a mixture including:
i) 18% to 98% by weight of at least one water-soluble excipient;
ii) 2% to 20% by weight of at least one water swellable polymeric material;
iii) 3% to 25% by weight of at least one water-insoluble hydrophobic inorganic salt that absorbs no more than 0.2% by water by weight at a relative humidity of 95% at 25° C.; and
iv) 3% to 25% by weight of at least one water-insoluble inorganic salt that absorbs between 0.3% and 3.0% water by weight at a relative humidity of 95% at 25° C.
with water to form wet granules;
b) drying the wet granules to form substantially dry granules;
c) milling the substantially dry granules to produce orally dissolving granules of a desired size; and
d) compressing the granules to form a tablet.

30. The method of claim 29 wherein the ratio of the at least one water-insoluble hydrophobic inorganic salt to the at least one water-insoluble inorganic salt is from about 1:10 to about 10:1.

31. The method of claim 29 wherein the particle size of the at least one water-insoluble hydrophobic inorganic salt and the at least one water-insoluble inorganic salt is no more than about 80 μm.

32. The method of claim 29 wherein the mean particle size of the substantially dry granules is from about 100 μm to about 200 μm.

33. The method of claim 29 further comprising adding a lubricant to the granules prior to compressing the granules into a tablet.

34. The method of claim 29 further comprising adding at least one active pharmaceutical ingredient.
